SEQUENCE LISTING (2 sequences)

SEQ ID NO: 1 - protein, 194 aa, Artificial
  HPV E6 Fusion Protein
NPVVGYIGERPQYRDLMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLGLFEAIAEFIEGGWEELIEG

SEQ ID NO: 2 - protein, 134 aa, Artificial
  HPV E7 Fusion Protein
NPVVGYIGERPQYRDLMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGLFEAIAEFIEGGWEELIEG

(19) United States
(12) Patent Application Publication
(10) Pub. No.: US 2011/0110901 A1
(43) Pub. Date: May 12, 2011

(54) METHODS OF LONG-TERM CULTURE OF EUKARYOTIC CELLS AND USES THEREOF

(76) Inventor: Chee Keong Choo, Hong Kong (CN)

(21) Appl. No.: 13/002,507

(22) PCT Filed: Jul. 17, 2009
(86) PCT No.: PCT/IB2009/053112

§ 371 Date: Jan. 4, 2011

Related U.S. Application Data
(60) Provisional application No. 61/081,740, filed on Jul. 18, 2008.

(51) Int. Cl. A61K 35/12; C07K 19/00; C12N 9/96; C12N 5/07; A01N 1/02; C12N 5/071; A61P 3/00; A61P 37/06; A61P 21/00; A61P 7/00; A61P 17/02; A61P 35/00; A61P 35/02; A61P 25/28; A61P 3/10

(52) U.S. Cl. 424/93.7; 530/350; 435/188; 435/375; 435/1.1; 435/325

(57) ABSTRACT

An immortalizing protein complex, comprises an internalizing molecule, a transforming polypeptide and an endosome releasing molecule; all molecule and/or polypeptides being operatively linked to one or more of the other polypeptides, and optionally one or more of a nuclear internalizing molecule and a telomere extending molecule. A method of producing immortalized cells or extending the life of primary cells, comprises contacting the immortalizing protein complex of the invention with a target cell under conditions and for a period of time effective for the protein complex to be internalized and the transforming polypeptide released, and allowing for the transforming polypeptide to extend the life of the cell and/or overcome cell arrest. Also provided are an immortalizing polynucleotide encoding the protein complex, a hybrid vector carrying an expression vector and the polynucleotide, a cell transformed with the hybrid vector, a cell expressing the protein complex, and a cell immortalization kit that comprises the protein complex and/or a polynucleotide encoding the protein and/or a hybrid expression vector carrying the polynucleotide, and instructions for its(their) use to practice the method for immortalizing cells. Cells and tissue transplants comprising the cell(s), and various uses for the immortalized cells and tissue thereof are also provided.

### FIELD OF INVENTION

This invention pertains to the field of immortalized cells, and their use in experimental research, therapeutics and testing. This invention provides a novel method of immortalizing cells that employs proteins instead of nucleic acids.

### BACKGROUND OF INVENTION

Conventional methods to produce transformed cells employ the DNA or RNA of E6/E7 viral oncogenes. Thereafter, a gene(s) may be transfected into a target cell. Alternatively, the gene(s) may be cloned into a viral vector, and the hybrid vector transferred into the target cells via viral infection. In many cases, the gene transfer vector will be tagged with a drug resistant gene to facilitate subsequent work steps and selection. Typically, the cells may be selected immediately upon gene transfer using a drug resistant gene. Alternatively, the normal cells may be allowed to die, and the surviving cells may be analyzed to determine their characteristics for future use.

There exists a compelling need for a method of producing cells in large quantities and lacking, or having minimal unwanted characteristics of cancerous and transformed cells. There is also a clear need for cell lines that may be easily derived, maintained in cultures and afford the ability of differentiating, genetic variation, stable metabolism, and consistent response to extracellular agents. For clinical application such as cell therapy and tissue engineering, it is pivotal to have scalable quantity of cells of various organ origins without genetic modification.

### SUMMARY OF INVENTION

For the practice of the invention, it is provided an immortalizing protein complex that comprises an internalizing molecule(s), a transforming polypeptide(s) and an endosome releasing molecule(s); in a preferred embodiment all molecules and/or polypeptides may be operatively linked to one or more of the other polypeptides. In another preferred embodiment, the internalization is endosome-mediated. In yet another preferred embodiment, the immortalizing protein complex further comprises a nuclear internalizing molecule(s) and a telomere extending molecule(s). In a further embodiment, the internalizing function and the endosome releasing function are performed by the same molecule and therefore the internalizing molecule and the endosome releasing molecule are of the same, such as the case in when viral particle and viral capsid proteins are used

Also part of the invention is an immortalizing polynucleotide(s), encoding the immortalizing protein complex(es); a hybrid vector carrying an expression vector(s), and the polynucleotide(s) of the immortalizing protein complex(es); a cell(s) that is transformed with the hybrid vector(s); a cell(s) that expresses the immortalizing protein complex(es); a composition having the immortalizing protein complex(es); and a cell immortalization kit, comprising the immortalizing protein complex(es) and/or a polynucleotide(s) encoding the protein complex(es) and/or a hybrid expression vector(s) carrying the polynucleotide(s), and instructions for its(their) use to practice the method for immortalizing cells.

In addition, another aspect of this invention relates to a method of producing immortalized cells or extending the life of primary cells, comprising

contacting the immortalizing protein complex(es) of the invention with a target cell(s) under conditions and for a period of time effective for the protein complex(es) to be internalized and the transforming polypeptide(s) to be released; and

allowing for the transforming polypeptide(s) to extend the life of the cell(s) and/or overcome cell growth arrest and/or overcome cell senescence and/or prevent cell differentiation. In a preferred implementation, the method further comprises removing the immortalizing protein complex from the cell culture media to reverse the immortalization effect to obtain cells with normal phenotype and capable of undergoing full differentiation.

This invention also relates to a cell(s) and tissues that have been treated according to the above method.

### BRIEF DESCRIPTION OF THE DRAWINGS

FIG. 1 shows a sodium dodecyl sulfate polyacrylamide gel electrophoresis (SDS-PAGE) of the expression upon induction of the E6 fusion protein complex according to one embodiment of the instant invention. Lane M contains a low molecular weight marker; Lane 1 contains the sample without IPTG induction incubated at 37° C. for 8 hrs; Lane 2 contains the sample induced by 0.5 mM IPTG at 37° C. for 4 hrs.

FIG. 2 shows an SDSD-PAGE and western blot of the E6 fusion protein complex according to the same embodiment of the present invention. Lane 1 shows a Coomassie Blue stained E6 fusion protein complex; Lane 2 shows a western blot of the fusion protein complex employing and anti-E6 antibody; Lane M contains a low molecule weight marker.

FIG. 3 shows a SDS-PAGE of an induced E7 fusion protein complex according to another embodiment of this invention. Lane M contains a low molecular weight marker; Lane 1 contains the supernatant induced by 0.5 mM IPTG at 37° C. for 4 hrs; Lane 2 shows the supernatant induced by 0.5 mM IPTG at 25° C. for 5 hrs; Lane 3 shows the supernatant without IPTG induction at 37° C. for 4 hrs.

FIG. 4 shows an E7 fusion protein complex after elution from an Ni-IDA column according to the same embodiment of the instant invention. Lane 1 shows the eluted fractions; Lane M shows a protein marker.

FIG. 5 shows the separation of E7 from the tag by Ni resin according to the same embodiment of the present invention. Lane M contains a MW marker; Lane 1 contains the wash from the column; Lane 2 shows a Western Blot after using HPV 16 E7 specific antibody.

FIG. 6 shows the response of keratinocytes to HPV 16 in the E6 and E7 fusion protein complexes according to another embodiment of this invention. (a) 0.75 μg/ml E6 FP+0.25 μg/ml E7 FP; (b) 1 μg/ml E6 FP+0.33 μg/ml E7 FP; (c) 0.67 to 2 μg/ml E7 FP; (d) >2 μg/ml E7 FP; (e) 2 to 6 μg/ml E6 FP; (f) >6 μg/ml E6 FP; (g) control.

Other objects, advantages and features of the present invention will become apparent to those skilled in the art from the following discussion.

### DETAILED DESCRIPTION OF THE INVENTION

This invention arose from a desire by the inventors to improve on prior art technology involving the use of primary cells and malignant and transformed cell lines for biomedical research. In their own research, the inventors were faced with the problems associated with the use of mortal primary cells that must be obtained anew from fresh cadavers and are difficult to maintain in culture. The inventors were also too familiar with the drawbacks of employing malignant and transformed cell lines in the laboratory, namely their lack of ability to differentiate, and the fact that they are prone to genetic mutation and aberrant metabolism and many times display an abnormal responses to extra-cellular agents.

In trying to overcome the above-mentioned drawbacks, the inventors discovered that employing a protein for the cell immortalization process they could produce cells of desired characteristics in large quantities and in a reproducible manner.

The present method is useful both in vivo, ex vivo and in situ applications. Exemplary uses include, but are not limited to, the production of extended life cells in culture, extension of stem cell and progenitor cell cultures and implants, extension of skin or other epithelial cell cultures and grafts, the expansion of mesenchymal cell cultures and grafts, and expansion of chondrocyte and osteocyte cultures and grafts. Exemplary stem and progenitor cells, whose lives are extended by the method of the invention include neuronal, hematopoietic, epithelial, pancreatic, hepatic, chondrocytic and osteocytic stem and progenitor cells, among many others. The present method is also applicable in wound and burn healing, and more generally in tissue repair applications, as well as cosmetic uses. The present invention is also applicable to prolonging the lifespan of a culture of normal cells or tissue generally being used for research purposes, and in one aspect to normal cells employed in secretion of therapeutic and other commercially significant proteins and products.

### Protein Based Cell Immortalization Method

The inventors are providing a process for manufacturing cell lines suitable for biological research and testing of foreign and endogenous agents that arose from their experimental work. In the process of the present invention, a gene may be expressed as a fusion protein using recombinant methods. The thus obtained fusion protein, existing in the form of a protein complex, comprises an immortalizing or transforming polypeptide, an internalizing or translocating polypeptide, and an endosome releasing signal(s). An optional addition to the protein complex is that of a cell surface receptor binding domain to aid in the internalization of the protein complex by the cell. The protein complex may also be produced as a fusion protein. The fusion protein may be generated post-transcriptionally, for example, by the chemical addition of the endosome releasing signal to the immortalizing protein, and optionally the addition of the receptor binding domain. Once obtained, the protein complex or the fusion protein is allowed to be internalized in a cell by spontaneous means, optionally by binding to the cell surface receptor via a receptor endocytosis pathway. The internalization is also optionally endosome-mediated. Once inside the cell, the protein complex or fusion protein enters the endosome, where the immortalizing polypeptide is separated from the rest of the protein or continues to be bound to the protein. As the endosome releasing signal disrupts the endosome, it releases the immortalizing protein, which then is free to exercise its immortalizing or growth promoting activity on the host cell.

This present invention is an effective process for prolonging the life of a cell and obtaining an immortalized cell line(s) employing a protein or protein complex rather than DNA or RNA encoding the immortalization gene to immortalize or extend the life span of the cell(s) in culture or in vivo. Although a specific example is provided, the method provided herein is also suitable for use with other immortalizing polypeptides and other types of cells. The benefits of cell lines developed using the novel methods are clear. The immortalized cells of this invention contain no permanent genetic alterations and, therefore, best resemble the original, primary cells. The protein complex is employed as an exogenous factor to be added to a cell, whether in culture or in vivo, to extend the cell life span for an extended period of time or indefinitely. It should be noted that the immortalizing effect may be reversed upon removal of the protein complex from the cell culture media. When this is done, the cells revert to their normal phenotype and may undergo full differentiation. The cells propagated using the method of this invention are far superior alternatives to conventional cell lines obtained by stimulation with immortalizing/translocation proteins that do not have the endosome release factor provided in the complex. Moreover, when the cells are propagated in a GMP environment they are suitable for human cell therapy and transplantation applications. This ability makes the present technology extremely important for the treatment of metabolic diseases, autoimmune diseases, congenital diseases, neuronal and muscular degenerative diseases, blood disorders, chronic wounds and ulcers, among many others. Examples of diseases that may be treated with the present cells are diabetes, autism, lupus, Alzheimer's disease, Huntington disease, hemophilia leukemia, lymphoma, burn and chronic wounds, among many others.

The invention will be described by reference to one or more examples, but is applicable across the board to many target cells and suitable immortalizing, transforming or internalizing polypeptides or proteins. The HPV 16 E6 and E7 oncogenes have been employed as examples of transforming proteins in the protein based immortalization of keratinocytes or keratinocyte life extension. The gene products of E6/E7 oncogenes of various subtypes of human papillomaviruses, SV 40 T antigen, Adenovirus E1a, and various growth promoting/immortalizing genes such as human telomerase reverse transcriptase (hTERT), ras, myc and others in combination are also suitable for use with the invention method to achieve similar results. In the formation of the fusion protein this invention also employs a cell internalizing polypeptide, e.g. EGF receptor recognizing domain. Other appropriate receptor recognizing domains should be employed for the immortalization or extension of life of other cells. Examples of receptors and cell combinations are VEGF receptor recognizing domain that would be suitable for endothelial cells and smooth muscle cells; IGFI and IGFII receptor recognizing domains that would be suitable for cardiac and skeletal muscle cells; c-kit receptor recognizing domain for heamatopoetic stem cells; and NFG receptor recognizing domain are for neurons and astrocytes, among many others known in the art. The fusion protein of the invention also comprises a domain of an endosome releasing signal such as the heamaglutinin peptide of the influenza virus, the entire viral capsid protein or even the entire virus.

Briefly, the HPV E6 protein is known to bind to the p53 tumor suppressor gene and causes its rapid degradation via the ubiquitin pathways. The thus treated cell will grow indefinitely for extended periods of time due to the absence of p53 protein. The HPV E7 protein, on the other hand, binds the pRB tumor suppressor gene and renders it incapable of sequestering the E2F growth promoting factor. The free E2F growth promoting factor promotes cell growth and avoids senescence. Under the influence of the E6 and E7 oncoproteins, the cells spontaneously acquire telomerase activity. It is important to note that the E6 and E7 oncoproteins of various HPV subtypes have the ability to bind to p53 and pRb, respectively. Nevertheless, since the E6 and E7 oncoproteins of HPV16 and HPV18 bind to their respective tumor suppressor gene with higher affinity than the rest, these proteins have the greater immortalizing or life extension ability in culture, as described in J Dermatol. 2000 Feb; 27(2):73-86. In a similar fashion, the SV40 T antigen and the Adenovirus E1a antigen are also extremely effective for immortalizing their target cells by interfering the p53 and pRB pathways, respectively.

The invention will now be described with reference to the claims. In a first aspect, this invention provides an immortalizing protein complex that comprises an internalizing molecule, optionally in the form of a protein or polypeptide; a transforming polypeptide and an endosome releasing polypeptide molecule that are operatively linked to one another, optionally linked to one another in the form of a fusion protein. In another preferred embodiment, the protein complex further comprises a nuclear internalizing molecule and a telomere extending molecule.

In one embodiment of the present invention, the internalizing molecule aids in transferring or internalizing the protein complex into the target cell(s). In one preferred embodiment, the internalizing molecule is endosome-mediated; yet in another preferred embodiment, the endosome-mediated internalizing molecule is receptor-mediated. In another preferred embodiment, the internalizing molecule(s) comprise(s) a cell-surface receptor binding molecule(s), a cell-surface internalizing molecule(s), and/or a virus binding receptor molecule(s). In a particularly preferred embodiment, the internalizing molecule(s) further comprise(s) an EGF receptor recognizing molecule or ligand, an IGF receptor recognizing molecule or ligand, a VEGF receptor recognizing molecule or ligand, a FGF receptor recognizing molecule or ligand, a PDGF receptor recognizing molecule or ligand, an insulin receptor recognizing molecule or ligand, a growth hormone receptor recognizing molecule or ligand, a hepatocyte growth factor receptor recognizing molecule or ligand, a chemokine receptor recognizing molecule or ligand, an erythropoietin receptor recognizing molecule or ligand, a cytokine receptor recognizing molecule or ligand, a cKit polypeptide, HSV virus protein VP22, and/or a liposome.

In another embodiment of the present invention, the transforming polypeptide is capable of overcoming cell differentiation and/or cell cycle arrest and/or cell senescence. In a preferred embodiment, the transforming polypeptide comprises one or more oncogene(s) expression product(s) and/or one or more telomere extending molecule(s). In a particularly preferred embodiment, the employed oncogene(s) expression product(s) may be a viral oncogene expression product(s) comprising one or more of HPV E6, HPV E7, SV40 T antigen(s), adenovirus E1a and adenovirus E1b, Epstein Barr virus, EBNA2 protein and LMP-1 protein and/or a cellular oncogene expression product(s) comprising one or more of ras, myc and src oncogene expression product(s). In another preferred embodiment, the telomere extending molecule comprises hTERT, among many others known in the art.

In yet another embodiment of the instant invention, the endosome releasing polypeptide promotes and/or facilitates the release of the transforming or immortalizing polypeptide from the endosome. In a preferred embodiment, the endosome releasing molecule(s) comprises an endosome releasing signal polypeptide(s). In a particularly preferred embodiment, examples of endosome releasing molecules include influenza viral envelope HA20, adenovirus capsid, adeno-associated virus viral capsid, retrovirus capsid, and other viral and synthetic endosome releasing molecules.

In still another preferred embodiment of this invention, the nuclear internalizing molecule comprises a nuclear receptor binding molecule(s) and/or a nuclear receptor molecule(s). In a preferred embodiment, examples of nuclear internalizing molecules include a steroid(s), a lipid-soluble moiety(ies), retinoic acid, vitamin D, members of the steroid receptor super family, retinoid receptors, and vitamin D receptor, or a combination(s) thereof.

In another embodiment of this invention, the telomere extending molecule(s) comprise(s) hTERT. In another embodiment, the telomere extending molecule comprises hTERT telomerase subunit, wherein the other two subunits of hTR and hTAP-1 are generally expressed in all cell types. On the other hand, for those cell types that hTR and hTAP-1 are not expressed therein, these two subunits can be introduced into the cells additionally. In one preferred embodiment, the telomerase extending molecule(s) comprise(s) the telomerase catalytic subunit(s), hTERT, hTR, hTR, hTP-A, or TERT of human origin or other mammalian homologues.

A specific immortalizing protein complex of the invention may have each polypeptide/molecule fused to at least one other polypeptide/molecule to form a fusion protein.

Another aspect of the present invention is an immortalizing polynucleotide that encodes the protein complex described above. Also provided herein in the form of a hybrid vector is an expression vector having operatively linked thereto the described polynucleotide. Also provided by the inventors is a cell transformed with the hybrid vector.

Yet another aspect, this invention provides a cell expressing the immortalizing protein complex.

In a further aspect, the aforementioned products may be packaged in the form of a cell immortalization kit, comprising, in the alternative or all inclusively, an appropriate immortalizing protein complex or fusion protein for its intended application and/or DNA or RNA segments encoding each domain of the protein complex and/or a polynucleotide encoding the entire protein complex and/or hybrid expression vectors, each carrying a nucleic acid segment encoding one polypeptide and/or a hybrid expression vector carrying the polynucleotide; and instructions for its use for immortalizing cells.

In another aspect, this invention also provides a composition, comprising the immortalizing protein complex or fusion protein of the invention, wherein different polypeptides may be substituted for each of the different domains.

Yet another aspect of the present invention disclosed and claimed in this invention is a method of immortalizing or of producing immortalized cells or extending the life of primary cells, which process comprises contacting the immortalizing protein complex of the invention with a target cell under conditions and for a period of time effective for the protein complex to be internalized and the transforming polypeptide released; and allowing for the immortalizing or transforming polypeptide to extend the life of the cell, and/or overcome cell growth arrest, and/or overcome cell senescence and/or prevent cell differentiation. In a preferred implementation, the method may comprise obtaining two separate immortalizing protein complexes, one comprising HPV E6 operatively linked to at least one endosome releasing molecule, and the other comprising HPV E7 operatively linked to at least one endosome releasing molecule, and incorporating both complexes into a target cell under conditions and for a period of time effective for the two protein complexes to be internalized, whereby the immortalizing or transforming polypeptides HPV E6 and HPV E7 may be released into the target cell, and allowing the extension of the life of the cell and/or overcoming or preventing cell arrest and/or overcome cell senescence and/or preventing cell differentiation.

In yet another implementation, the method further comprises removing the immortalizing protein complex from the cell culture media to reverse the immortalization effect to obtain cells with normal phenotype and capable of undergoing full differentiation.

The target cells for immortalization may be of many different types, and the method of the present invention is effective in prolonging the life of numerous cells, and it may be employed for the treatment of any and all primary cells as target cells. In one implementation, the types of cells comprises islet cells, hepatocytes, keratinocytes, endothelial cells, smooth muscle cells, skeletal and cardiac muscle cells, nerve cells. astrocytes, and adult and cord blood hematopoetic cells. However, many others are also suitable for treatment by the method of this invention. In another preferred implementation, the immortalizing protein complex is provided in the form of a liposome or any one of many other known encapsulating entities.

In one implementation of the method disclosed in this invention, the fusion protein or polypeptide segment(s) of any one, or any combination, of elements of the immortalizing protein complex may be obtained by growing feeder cells carrying a hybrid vector(s) expressing the immortalizing protein complex or any one of the immortalizing or transforming polypeptides, followed by contacting the hybrid vector(s) with the target cells. In an alternative implementation, the immortalizing protein complex is further engineered to have a secretion signal so that they will be secreted from the feeder cells to the growth medium and allow the protein complex to enter the target cells via various protein internalization approaches. The feeder cells may be maintained separately from the target cells so that the feeder cells may be removed from the medium, as is known in the art. Examples of feeder cells employed include 3T3, or stromal cells. Under certain circumstances, the feeder cells themselves may be placed in the same medium as the target cells, such as co-culturing of these two types of cells, the removal of the feeder cells from the medium may be necessary. On the other hand, the immortalizing protein complex may also be required to be separated from the target cells and removed from the medium when the target cells are to be reverted to their normal phenotypes.

Thus, in one implementation, the feeder cells may be co-cultured with the target cells. In another, the feeder cells are maintained separately from the target cells so that the feeder cells may be removed from the medium. In a preferred implementation of the method, the internalization of the immortalizing protein(s) into the target cell(s) may be aided by electroporation, microinjection, transfection, and/or nanoparticle bombardment, and/or other techniques known in the art. Specifically, in nanoparticle bombardment, the nanoparticles are pre-coated or conjugated with the necessary components of the immortalizing protein complex. The nanoparticales are then being bombarded into the cells using machical force such as the Helio gun manufactured by BioRad.

The cells obtained by the method described above are suitable for many uses, whether for experimental research, as an alternative to clinical testing, for expansion of freshly obtained primary cells, or for cell and tissue therapy. The cells may be employed by themselves or by means of a device or implant to preserve cell isolation and sterility. One aspect of the present invention is in a method of reproducing organ or function specific cells by obtaining a desired organ or function specific target cell(s); immortalizing the organ or function specific cells by the method of the invention; growing and expanding the thus immortalized cells; and re-mortalizing the expanded cells by withdrawing the immortalizing protein complex or fusion protein from the medium.

In one implementation, the organ or function specific cells may be multipotent stem cells. In another implementation, the organ or function specific cells may comprise bone marrow mesenchymal stroma cells, cardiac muscle cells, islet of Langerheim cells, liver cells, brain cells, muscle cells, skin cells, kidney cells, bone cells, stem cells, hematopoietic cells, pancreatic cells, thyroid gland cells, hearing associated cells, vision associated cells, olfactory cells, and other cells. In yet another implementation, the target cells may comprise dividing or resting cells, terminally differentiated starting organ cells, or the target cells may be transformed while being immortalized. The organ or function specific cells may comprise autologous or allogeneic cells as well.

Another aspect of this invention also provides a cell(s) obtained by the method of the invention, whether stopping at the end step of cell immortalization, or at the further amplification of the immortralized cells, or at the re-mortalization of the amplified cells.

This invention, in one aspect, also provides a method of transplanting cells for organ regeneration, comprising transplanting normal, functional organ or function specific cell(s) obtained by the method of the invention, into a subject's organ site under conditions effective for continued growth; and allowing the cells to grow and regenerate the organ.

In yet another aspect provided herein is a method of treating a disease or condition associated with an organ or function specific cell malfunction. Such method may comprise obtaining a targeted normal cell(s) from the specific organ or function, immortalizing it(them) by the method of the instant invention employing the appropriate immortalizing protein domains, and transplanting the cells with or without prior re-mortalization as described above. The method of this invention may be implemented in the treatment of many diseases or conditions. Examples, in one implementation, are those diseases and conditions that comprise or are associated with metabolic diseases, e.g. diabetes, heart disease, burns, wounds, blindness, some types of hearing impediments, liver diseases, autoimmune diseases, blood disorders including hemophilia and neoplasms, among many others.

The present patent in another aspect provides a cell tissue transplant, comprising cells obtained by the process of the invention. The tissue cells may originate from different types of target cells, as described above.

In this context, the artisan will find simple and comprehensive guidance in this patent for obtaining cells and tissue transplants comprising the cell(s) that are suitable for many specific applications. Yet in another aspect of this invention, a method is provided of regenerating an organ by transplanting normal, functional organ or function specific cell(s) or cell tissue, obtained by the method described above, into a subject under conditions effective for continued growth; and allowing the cells to grow and regenerate, or generate a functional organ.

### Fusion Proteins Employed in the Examples

### 1.E6 Fusion Protein

Amino Acid Sequence
(SEQUENCE ID NO. 1)
(EGFR binding domain) (Human Papillomavirus 16 E6 oncoprotein)
NPVVGYIGERPQYRDL-MHQKRTAMFQDPQERPRKLPQLCTELQTTIHDIILECVYCKQQLLRREVY
DFAFRDLCIVYRDGNPYAVCDKCLKFYSKISEYRHYCYSLYGTTLEQQYNKPLCDLLIRCINCQKPL
CPEEKQRHLDKKQRFHNIRGRWTGRCMSCCRSSRTRRETQL-GLFEAIAEFIEGGWEELIEG
(Endosome releasing signal)

### 2.E7 Fusion Protein

Amino Acid Sequence
(SEQUENCE ID NO. 2)
(EGFR binding domain) (Human Papillomavirus 16 E7 oncoprotein)
NPVVGYIGERPQYRDL-MHGDTPTLHEYMLDLQPETTDLYCYEQLNDSSEEEDEIDGPAGQAEPDR
AHYNIVTFCCKCDSTLRLCVQSTHVDIRTLEDLLMGTLGIVCPICSQKP-GLFEAIAEFIEGGWEELIE
G
(Endosome releasing signal)

### REFERENCES

### Mol Cells. 2002;13(3):351-61.

### J. Dermatol. 2000; 27(2):73-86.

U.S. Pat. No. 6,339,139 to Gu, et al.
U.S. Pat. No. 5,635,383 to Wu, et al.
PCT Publication No. WO 00/031238
US Patent Application No. US 2005/0244969
J. Biol. Chem. (1988) 15:263(29) 14621-4

Having now generally described this invention, the same will be better understood by reference to certain specific examples, which are included herein for purposes of illustration only and are not intended to be limiting of the invention or any embodiment thereof, unless so specified.

### EXAMPLES

### Example

### Subcloning of an E6 Fusion Gene into the pET15b Vector

An E6 fusion polynucleotide (SEQUENCE ID NO. 1), that comprises from the 5′—to the 3′—end a DNA segment encoding a polypeptide comprising an EGF receptor recognizing domain, the HPV 16 E6 oncogene, and a DNA encoding an endosome releasing signal polypeptide, was synthesized and cloned into a pET15b vector (Novagen, Cat. No. : 70755) at the Nco I and Xho I sites. The fusion polynucleotide was provided with a his-tag at the 5′ terminus.

The thus obtained hybrid pET15b carrying the E6 fusion polynucleotide was subjected to DNA sequencing to verify its sequence.

### Example 2

### Transformation of E. coli BL21

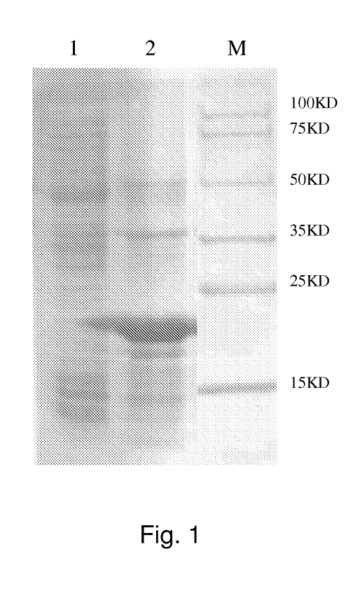

Five nanograms of the hybrid plasmid pET15b carrying the E6 fusion polynucleotide were transformed into E. coli BL21 and allowed to grow. Thereafter, one colony was selected, and cultured in 4 mL LB broth (100 ug/ml Amp) and expression was induced employing 0.5 mmol/L IPTG The expression products were detected by sodium dodecyl sulfate polyacrylamide gel electrophoresis, e.i. 15% SDS-PAGE. The results are shown in FIG. 1 accompanying this patent.

### Example 3

### Protein Expression

A sample of the E. coli BL21 strain was cultured in 10 L LB media for 4 hours, and then induced with 0.5 mmol/L IPTG for 4 hours. The cells were harvested by centrifugation, and the resulting cell paste was resuspended in 1000 ml lysis buffer and then sonicated on ice to obtain a cell lysate.

### Example 4

### Protein Purification

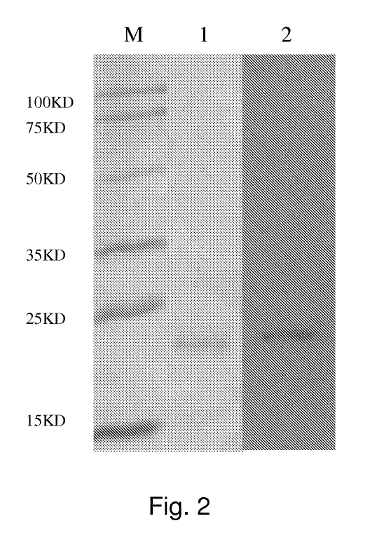

After sonication, the lysate was centrifuged at high speed for 10 minutes, and the precipitate collected. The precipitate was resolved by gel electrophoresis loading it with a 5× loading SDS buffer on a 15% SDS-PAGE gel, and the gel was then negatively stained. The target band was cut and electroeluted as shown in FIG. 2 accompanying this patent. The identity of the bands was verified by Western Blot. See, also FIG. 2.

The buffer employed for the final solution was 20 mM Tris-HCl (pH8.0), and the target protein concentration was found to be 0.633 mg/ml. The total volume of the lysate was 100 mL.

### Example 5

### Subcloning of the E7 Fusion Polynucleotide into a pET32a Plasmid

The E7 fusion polynucleotide (SEQUENCE ID NO. 2) (5′-EGF receptor recognizing domain-E7 oncogene-endosome releasing signal-3′) was synthesized and cloned into a pET32a plasmid (Novagen, Cat. No:69015) using the Kpn I and Not I enzyme recognition sites. The sequence of the resulting hybrid pET32a-E7 fusion plasmid was verified by DNA sequencing.

### Example 6

### Transformation of E. coli BL21

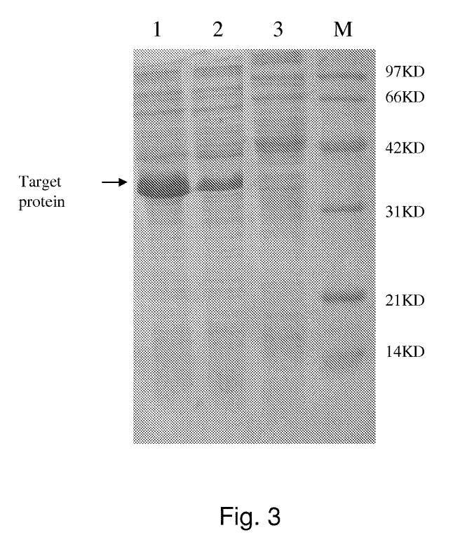

Five nanograms hybrid pET32-E7 fusion polynucleotide plasmid were transformed into E. coli BL21 cells. One cell colony was selected, cultured in 4 mL LB broth (100 ug/ml Amp), and induced with 0.5 mmol/L IPTG The expression results were observed after separation by gel electrophoresis in 15% SDS-PAGE. See, FIG. 3 provided with this patent.

### Example 7

### Protein Expression

A sample of the E. coli BL21 strain was cultured in 10 L LB media for 4 hours, and then induced with 0.5 mmol/L IPTG for 4 hrs. The cells were harvested by centrifugation, and the thus resulting cell paste was resuspended in 1000 ml lysis buffer and then sonicated on ice.

### Example 8

### Protein Purification

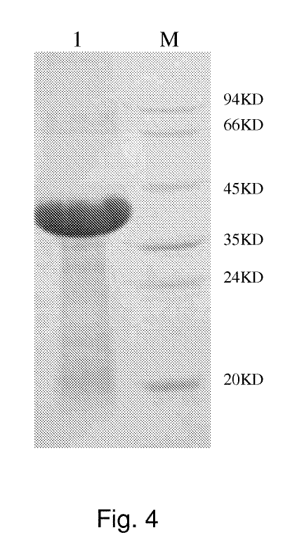

The sonicated lysate was centrifuged at high speed for 10 minutes, and the supernatant was collected and loaded onto an Ni-IDA column for purification. Samples of the eluting fractions were subjected to gel electrophoresis on 15% SDS-PAGE. See, FIG. 4. The fractions containing target protein were collected and dialysed against 20 mM Tris-HCl (pH 8.0).

### Example 9

### Protein Digestion and Separation

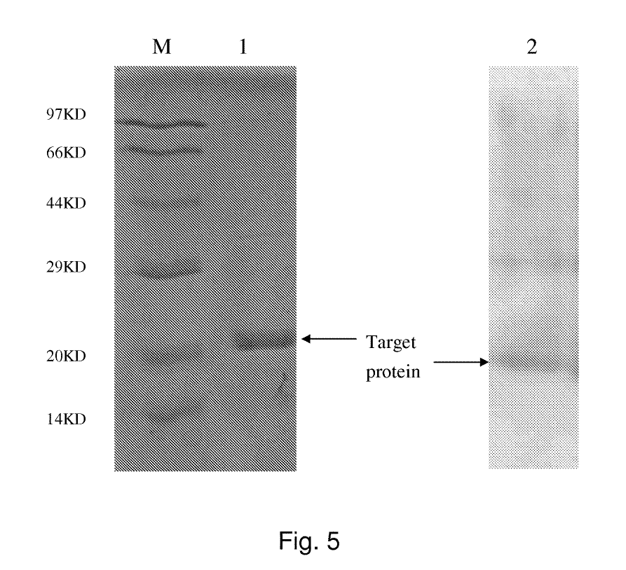

200 mg of protein was digested with 6000 units of EK for 10 hours and then separated by NI-IDA column, the flow through was collected, the column was then washed with wash buffer, and eluted with elute buffer. The target protein found in the washing buffer was verified by Western Blot (See, FIG. 5 of this patent. The final protein concentration was about 1 mg/ml (in 20 mM Tris, pH 8.0), and the final volume was 50 ml.

### Example 10

### Culture of Immortalized Cells

Normal human keratinocytes were purchased from Gibco (Gaithersburg, USA, Cat # 12568-010). The cells were isolated from a pool of male neonates' foreskins The cells were propagated in complete Keratinocyte SFM medium (Gibco, Gaithersburg, USA, Cat # 17005-042), supplemented with 1:100 dilution of penicillin-Streptomycin (Gibco, Gaithersburg, USA, Cat # 15140-122).

The frozen cells were quickly thawed and grown in a 25 cm² culture vented flask. When the keratinocytes had grown to 80% confluence they were passaged using mild Trypsin-EDTA digestion. Briefly, a cell monolayer was rinsed with 4 ml of HBSS (Gibco, Gaithersburg, USA, Cat # 14170-112), and incubated at room temperature with 4 ml of Trypsin-EDTA (Gibco, Gaithersburg, USA, Cat # 25300-054) diluted with Versene (Gibco, Gaithersburg, USA, Cat # 15040-066) at a 1:10 ratio.

Once the cells were detached from the flask, they were neutralized with 2 ml of HBSS containing 10% calf bovine serum (Gibco, Gaithersburg, USA, Cat # 26170-035), and centrifuged at 10×g for 5 minutes. The supernatant was discarded, and the cell pellet was re-suspended in complete growth medium at a 1:4 splitting ratio. On average the cells took about 10 days to reach 80% confluence again, with the doubling time of the cells being approximately 5 days.

### Example 11

### Results from Culture of Cells with E6 Fusion Protein

Cells were seeded at 20% confluent and cultured in 5 ml of Keratinocyte SFM medium at 37° C. in 25 cm² culture flasks. After two months of culture, a dose response curve to each of the fusion proteins was performed on the keratinocytes. Surprisingly, the E6 fusion protein proved to be lethal to the cells at high concentrations. Although being intended only as a hypothesis, this effect may be due to the cells competing with EGF for binding to the EGF receptor.

Cell death was observed immediately the next day if the concentration was about 6 μg/ml E6 fusion protein or higher. At a lower concentration, e.g. at about 2 μg/ml to about 6 μg/ml, cell death was observed to occur only after approximately 5 days. No cell death was observed at a concentration of or lower than about 1.5 μg/ml E6 fusion protein, when the cells were observed for a period of more than 4 months.

### Example 12

### Results from Culture of Cells with E7 Fusion Protein

Cells were seeded at 20% confluent and cultured in 5 ml of Keratinocytes SFM medium at 37° C. in 25 cm² culture flasks. After two months of culture, a dose response curve of the fusion protein was performed on the keratinocytes. Surprisingly, the E7 fusion protein also proved to be lethal to the cells at high concentrations. As already being indicated above, this may be due to the fact that the cells were competing with EGF for binding to the EGF receptor.

For the E7 fusion protein acute cell toxicity was seen at about 2 ug/ml E7 fusion protein. Mid-term toxicity was observed at concentrations of 0.6 μg/ml to less than about 2 μg/ml E7 fusion protein. At concentration at or lower than about 0.67 μg/ml E7 fusion protein no cell toxicity at all was encountered when the cells were observed for a period of 4 months.

### Example 13

### Long Term Culture of Cells with E6 Fusion Protein

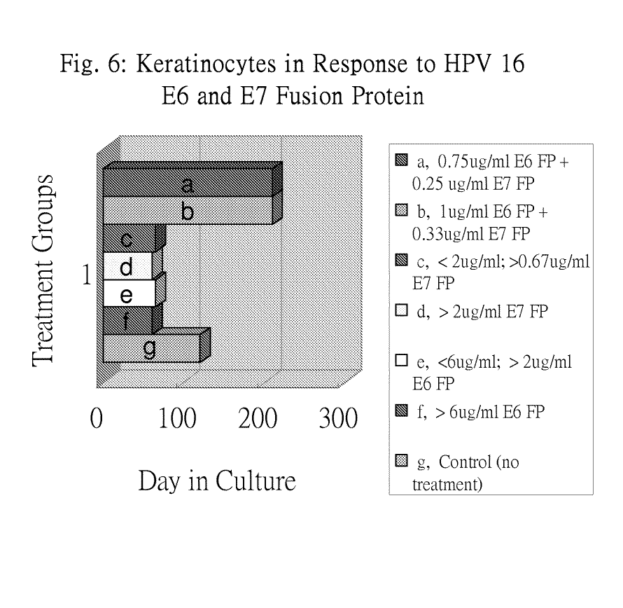

The following concentrations of the fusion proteins were tested in long term experiments: 1 ug/ml and 0.75 ug/ml of E6 fusion protein combined with 0.33 ug/ml and 0.25 ug/ml of E7 fusion protein, respectively. The results were summarized in FIG. 6 accompanying this patent.

In summary, the keratinocytes without treatment started to senesce after 4 months whereas the treated keratinocytes with non lethal dose of HPV 16 E6 and E7 continued to propagate up to 7 months. After 7 months their growth was significantly slowed down; even though the cells remained viable but the doubling time was prolonged to 15 days or more and the experiment was terminated.

### Example 14

### Lipid Soluble Nuclear Internalizing Molecule

In another aspect of the present invention, the immortalizing polypeptide is adapted to be linked or conjugated to a lipid soluble nuclear receptor ligand or a lipid soluble moiety. The nuclear receptor ligand is selected based on the expression of its corresponding nuclear receptor in the target cells.

In this application, the lipid soluble nuclear receptor ligand or the lipid soluble moiety will diffuse across the cell surface membrane, bringing along the immortalizing polypeptides into the cytoplasm of the target cells. Specifically, when the nuclear receptor ligand is employed, it will bind to the corresponding nuclear receptor in the cytoplasm, dimerize and translocate into the cell nucleus. Alternatively, when lipid soluble moiety is used, it will enter the nucleus from the cytoplasm by diffusing through the nuclear membrane, and thus the immortalizing polypeptide is tagged along into the nucleus.

The invention and some preferred embodiments and examples thereof now being fully described, it will be apparent to one of ordinary skill in the art that many changes and modifications can be made thereto without departing from the spirit or scope of the invention as set forth herein. Although the above description refers to particular embodiments, it will be clear to one skilled in the art that the present invention may be practiced with variation of these specific details. Hence this invention should not be construed as limited to the embodiments set forth herein.

For instance, multiple molecules may be used simultaneously as the transforming polypeptides, as in the case of the HPV E6 and HPV E7 polypeptides. As such, it is clear that multiple expression vectors may be employed to express the transforming polypeptides separately for generating the immortalizing protein complex.

What is claimed is:

1. An immortalizing protein complex, comprising
at least one internalizing molecule;
at least one transforming polypeptide;
at least one endosome releasing molecule; and
wherein said at least one molecule or polypeptide is operatively linked to at least one said molecule or polypeptide.

2. The immortalizing protein complex of claim 1, further comprising at least one nuclear internalizing molecule and at least one telomere extending molecule.

3. The immortalizing protein complex of any one of claims 1 to 2, wherein
said transforming polypeptide is capable of overcoming cell senescence; and/or
said endosome releasing molecule promotes release of said transforming polypeptide from the endosome; and/or
said internalizing molecule aids in transferring said immortalizing protein complex into target cell(s).

4. The immortalizing protein complex of any one of claims 1 to 2, wherein said internalizing molecule is endosome-mediated.

5. The immortalizing protein complex of claim 4, wherein said internalizing molecule is receptor-mediated.

6. The immortalizing protein complex of claims 5, wherein said internalizing molecule comprises a cell-surface receptor binding molecule(s), a cell-surface internalizing molecule(s) and/or a virus binding receptor molecule(s).

7. The immortalizing protein complex of claim 6, wherein the internalizing molecule comprises an EGF receptor recognizing molecule(s) or ligand(s), an IGF receptor recognizing molecule(s) or ligand(s), a VEGF receptor recognizing molecule(s) or ligand(s), a FGF receptor recognizing molecule(s) or ligand(s), a PDGF receptor recognizing molecule(s) or ligand(s), an insulin receptor recognizing molecule(s) or ligand(s), a growth hormone receptor recognizing molecule(s) or ligand(s), a hepatocyte growth factor receptor recognizing molecule(s) or ligand(s), a chemokine receptor recognizing molecule(s) or ligand(s), an erythropoietin receptor recognizing molecule(s) or ligand(s), a cytokine receptor recognizing molecule(s) or ligand(s), a c-kit polypeptide, HSV virus protein VP22 and/or a liposome(s).

8. The immortalizing protein complex of any one of claims 1 to 2, wherein said transforming polypeptide comprises one or more oncogene(s) expression product(s) and/or one or human telomerase reverse transcriptase (hTERT).

9. The immortalizing protein complex of claim 8, wherein said oncogene(s) expression product(s) comprise(s)
a viral oncogene expression product(s) comprising one or more of HPV E6, HPV E7, SV40 T antigen(s), adenovirus E1a, adenovirus E1B, Epstein Barr virus, EBNA2 protein and LMP-1 protein; and/or
a cellular oncogene expression product(s) comprising one or more of ras, myc and src oncogene expression product(s).

10. The immortalizing protein complex of any one of claims 1 to 2, wherein said endosome releasing molecule comprises an endosome releasing signal polypeptide(s).

11. The immortalizing protein complex of claim 10, wherein said endosome releasing molecule comprises a viral or synthetic endosome releasing molecule(s).

12. The immortalizing protein complex of any one of claims 1 to 2, wherein said nuclear internalizing molecule comprises a nuclear receptor binding molecule(s) and/or a nuclear receptor molecule(s).

13. The immortalizing protein complex of claim 12, wherein said nuclear internalizing molecule further comprises a steroid(s), a lipid-soluble molecule(s), retinoic acid, vitamin D, members of steroid receptor super family, members of retinoid receptor family and vitamin D receptor or a combination(s) thereof.

14. The immortalizing protein complex of claim any one of claims 1 to 2, wherein said telomere extending molecule comprises hTERT.

15. The immortalizing protein complex of claim 14, wherein said telomere extending molecule further comprises a telomerase catalytic subunit(s) hTERT, or hTR, hTP-A, TERT of human origin or other mammalian homologue.

16. A method for immortalizing cells, comprising
contacting said immortalizing protein complex of any one of claims 1 to 2 with a target cell under conditions and for a period of time effective for said immortalizing protein complex to be internalized and said transforming polypeptide to be released; and
allowing for said transforming polypeptide to extend the life of the cell and/or overcome cell arrest, and/or overcome cell senescence and/or prevent cell differentiation.

17. The method of claim 16, further comprising removing said immortalizing protein complex from the cell culture media to reverse the immortalization effect to obtain cells with normal phenotype and capable of undergoing full differentiation.

18. The method of claim 16, wherein said target cells comprise keratinocytes, hepatocytes, islet cells, cardiac myocytes, skeletal myocytes, endothelail cells, smooth muscle cells, neuronal cells and astrocytes.

19. The method of claim 16, wherein said immortalizing protein complex is contacted with said target cell(s) in the form of a liposome.

20. A method of reproducing organ or function specific cells, comprising
obtaining a desired organ or function specific target cell(s);
immortalizing the organ or function specific cells by the method of claim 16;
growing and expanding the thus immortalized cells; and
remortalizing the expanded cells by withdrawing the immortalizing protein complex from the medium.

21. A method of transplanting a cell(s) or cell tissue for organ regeneration or generation , comprising
transplanting the normal, functional organ or function specific cell(s) obtained by said method of claim 20, into a subject under conditions effective for its(their) continued growth; and
allowing said cell(s) to grow and regenerate or generate the organ.

22. A method of treating a disease or condition associated with an organ or function specific cell(s) malfunction, comprising transplanting the cell(s) obtained by said method of claim 20 into a subject in need thereof.

23. A tissue transplant, comprising the cell(s) obtained by said method of claim 20.
